(19) United States
(12) Patent Application Publication
(10) Pub. No.: US 2020/0253870 A1
(43) Pub. Date: Aug. 13, 2020

(54) A LDV LIPOSOMAL FORMULATION OF PHOTO SYSTEM-I FOR TREATMENT OF CANCER

(71) Applicant: COUNCIL OF SCIENTIFIC AND INDUSTRIAL RESEARCH, New Delhi (IN)

(72) Inventors: Surajit GHOSH, Kolkata (IN); Abhijit SAHA, Kolkata (IN); Subhajit GHOSH, Kolkata (IN); Saswat MOHAPATRA, Kolkata (IN); Batakrishna JANA, Kolkata (IN); Debmalya BHUNIA, Kolkata (IN)

(21) Appl. No.: 16/339,759

(22) PCT Filed: Sep. 16, 2017
(86) PCT No.: PCT/IN2017/050405

§ 371 Date: Apr. 5, 2019

(30) Foreign Application Priority Data
Oct. 5, 2016 (IN) 201611034058

(51) Int. Cl. A61K 9/127; A61K 47/69; A61K 36/00

(52) U.S. Cl. CPC A61K 9/1271; A61K 36/00; A61K 47/6911

(57) ABSTRACT

Present invention relates to a formulation from natural product. The formulation is LDV liposomal formulation of Photo System-I (PSI) which causes apoptotic death of cancer cells through ROS generation and a simple way for targeted delivery of the said formulation specifically to melanoma cancer cell. LDV liposomal formulation of PSI was prepared for targeted delivery to melanoma cancer cell. Through this targeted delivery inventors have shown that LDV liposomal formulation of PSI specifically kills melanoma cancer cells leaving normal cells unaffected. The present invention elucidates that LDV liposomal formulation of PSI is more potent anti-cancer agent than doxorubicin for melanoma.

### FIELD OF THE INVENTION

The present invention provides LDV-liposomal formulation of Photosystem I (PSI) useful for anti-cancer therapy. More specifically the present invention provides LDV liposomal formulation of PSI, which specifically targets as well as kills B16F10 (mouse melanoma) and A375 (human melanoma) cancer cells without affecting the WI38 (normal lung fibroblast) cell. The PSI in the LDV liposomal formulation is isolated from Indian spinach leaves, Spinacia oleracea.

### BACKGROUND OF THE INVENTION

In present scenario, cancer therapy faces major problem as most of the drugs are poor water soluble and lack of specificity towards certain cancer cells, causing toxicity to normal cells and resulting in various side effects in our body such as hair loss, thickening of the skin, redness or swelling on the palms of the hands, headache, fever etc. Drugs available for melanoma viz. docetaxel, dabrafenib, cobimetinibetc are generally small chemical compounds, poor water soluble, non-specific in nature and exerting toxic effect on our body as they kill normal cells along with cancer cells. Thus, developments of highly biocompatible (water soluble) and cancer cell specific drugs or drug formulation are extremely necessary in current scenario of cancer drug development. Another important fact is that most of the anticancer drugs are not easily available in market and are highly expensive, which are un-affordable for poor patients. Therefore, development of anti-cancer drugs from easily available natural source is highly demanding and extremely important. So, drugs obtained directly from natural source will be less expensive, easily available and affordable for poor peoples.

There are excellent source of bioactive components in plant and exerting their beneficial effect. Therefore, natural plants are used for the treatment of various diseases (H. Wang, et al. Anticancer Agents Med Chem., 2012, 12, 1281-1305) including cancer. Many natural products are used as anti-cancer and chemo preventing agents. Chemo preventing agents from edible phytochemicals control upstream signals that lead to genotoxic damage, redox imbalances and other forms of cellular stress and considered to be a readily applicable, acceptable and inexpensive approach for controlling cancer (G. A. Karikas, J. Buon., 2010, 15, 627-38).

Medicinal products from nature have been used for therapeutics against various diseases including cancer from ancient days. In modern chemotherapy, around 75% of the anticancer agents were derived from natural products (D. J. Newman, et al. Journal of Natural Products, 75, 311-335, 2010; G. M. Cragg, et al. Journal of Ethno pharmacology, 100, 72-79, 2005; M. A. Jordan, et al., Cancer Research, 51, 2212-2222, 1991; L. Nobler, et al. Cell Biol., 68, 1344-1351, 1990). But, there is a problem to use of the active component as a drug because of solubility problem and targeting issues. The active component can kill the non-cancerous/normal cells also.

A light harvesting pigment protein complex was isolated from plant leaves and it was found that the protein complex is structurally and functionally similar to Photosystem I (J. E. Mullet, et al. Plant Physiol., 65, 814-822, 1980). This protein complex was named as native PSI complex or simply PSI. This report describes only about the characterization of PSI. There are many studies regarding isolation technique of PSI (P. Kiley, et al. PLoS Biology, 3(7), e230, 2005; M. Hussels, et al. FEBS Letters, 585, 2445-2449, 2011; J. Liu, et al. Photosynth Res., 112, 63-70, 2012; X. Yan, et al. Langmuir, 28, 15080-15086, 2012). Photosystem I (PSI) is a large multi-protein light-harvesting complex (A. Amunts, et al. Nature, 447, 58-63, 2007), which generates the most negative red-ox potential and reactive oxygen species (ROS) in plant (S. E. Tjus, et al. Plant Physiol., 125, 2007-15, 2001). But, this report did not include the application of ROS generation for cancer therapy. Recently, various ROS generators have been used as anticancer agents in various cancer cells as well as chemo-sensitive enhancer against drug resistant cancer cell lines (X. Mao, et al. Cell Res., 18, 879-88, 2008). But, none of them are targeted specifically to cancer cells without affecting normal cells. All of them are not easily obtained from plant and are not easily available in market. Many of them are not soluble in water. It has been reported that PSI and PSII generates ROS and it can be used to act as anticancer agent (F. Bellemare, et al. PCT/CA2010/000094, 2010). But, this report does not show any experiment on the anti-cancer property of PSI. This report only proposed that PSI can be used for sensitizing the tumor cell. Further targeted cancer therapy using PSI was not shown in any previous report. Although, liposomal formulation of PSI is reported for stabilizing PSI (P. C. Pennisi, et al. Cellular and Mol., Bioengg., 2, 156-165, 2009), where only preparation of liposomal PSI was reported without showing the application of liposomal PSI for any kind of use. Again, it has been described on simple liposomal formulation. It did not show preparation of targeted liposomal PSI formulation. In addition, till now melanoma cell targeted liposomal formulation of PSI was not known. Although, LDV a tripeptide, is known to bind with α4β1-integrin receptor of cancer cell (S. Zhong, et al. Pharmaceutical Research, 26, 2578-2587, 2009) but preparation of LDV-liposome and its application for targeting α4β1-integrin receptor of cancer cell is not known.

Taking together, from these knowledge gap we have developed a novel formulation which can be used as an affordable and low cost melanoma therapy.

### OBJECTIVES OF THE INVENTION

The main object of the present invention is to provide LDV-liposomal formulation of PSI from natural sources wherein the ratio of PSI to LDV tri-peptide is in the range of 1:100 to 1:400.

Another object of the present invention is to provide LDV-liposomal formulation of PSI for targeted delivery by selectively targeting α4β1 integrin receptor on melanoma cells.

Yet another object of the present invention is to provide easily available potent anti-cancer drug at low cost with low side effects.

### SUMMARY OF THE INVENTION

The present invention provides a novel LDV liposomal formulation of photosystem-I (PSI) for anticancer therapy, wherein the ratio of PSI to LDV long chain, a short tri-peptide with C-16 long chain, is in the range of 1:100 to 1:400.

In an embodiment of the present invention, the liposome comprises of LDV long chain, DOPC (1,2-Dioleoyl-sn-glycero-3-phosphocholine) and cholesterol in a ratio of 1:1:0.5 respectively.

In an embodiment of the present invention, the percentage viability of B16F10 is in the range of 2% to 80% and of A375 melanoma cancer cells is in the range of 0.5% to 94%.

In another embodiment of the present invention, the IC₅₀ value of the said formulation is found to be in range of 3.98 to 6.01 μg/mL for B16F10 and A375 melanoma cancer cells.

In another embodiment of the present invention, the LDV liposomal formulation of PSI is useful for causing 83.3% and 96.3% apoptotic death of B16F10 and A375 cancer cells respectively at a concentration of 15 μg/mL.

In yet another embodiment, the present invention provides a method of preparation of the LDV liposomal formulation of PSI, wherein the composition comprises of the following steps of:
a) mixing chloroform in a glass vial, with LDV-C16 long chain (1 mM), DOPC lipid (1 mM) and cholesterol (0.5 mM) to obtain a solution,
b) removing chloroform from the mixture obtained in step (a) followed by drying the vial by evaporation using nitrogen flush,
c) drying the vial under vacuum for 2 hours to obtain dried mixture,
d) adding PSI to the dried mixture obtained in step (b) followed by incubation for a period in the range of 6 to 8 h at 4° C. and incubating it over-night, at 4° C.,
e) vortexing the solution in the vial by bath sonicating and probe sonicating.

In another embodiment of present invention, the prepared composition obtained from an isolate of an edible plant is less toxic compared to the anti-cancer drugs selected from a group of doxorubicin, dacastinib, etc.

### ABBREVIATION

PSI: Photosystem I.

ROS: Reactive oxygen species.

DOPC: 1, 2-Dioleoyl-sn-glycero-3-phosphocholine.

MTT: Methyl thiazolyl diphenyl-tetrazolium bromide

PBS: Phosphate Buffer Saline

FACS analysis: Fluorescence activated cell sorting analysis

HBUT: N, N, N′, N′-Tetramethyl-O-(1H-benzotriazol-1-yl) uroniumhexafluorophosphate

DMF: N, N′-Dimethyl formamide

DCM: Dichloromethane

DMSO: Dimethyl Sulfoxide

MeOH: Methanol

EtOH: Ethanol

rTdT: Recombinant Terminal Deoxynucleotidyl Transferase

RNase A: Ribonuclease A

### BRIEF DESCRIPTION OF THE DRAWINGS

FIG. 1: Cell viability of LDV liposomal PSI in B16F10.

FIG. 2: Comparison with doxorubicin in A375 cell. (a) Cell viability assay of A375 cells with LDV liposomal formulation of PSI shows IC50 at 6.01 μg/mL. (b) Cell viability assay of A375 cells with doxorubicin shows IC50 at 10.19 μg/mL.

FIG. 3: Cell viability study of 15.62 μg/mL concentration of LDV liposomal PSI and doxorubicin in B16F10 and WI38 cell lines.

FIG. 4: Apoptotic death of B16F10 cells. (a) Control B16F10 cells. (b) Cells were treated with 15 μg/mL concentration of doxorubicin. (c) Cells were treated with 15.62 μg/mL concentration of LDV liposomal formulation of PSI. (d) Comparative bar diagram of apoptotic death after treatment with 15 μg/mL concentration of doxorubicin and LDV liposomal PSI.

FIG. 5: Apoptotic death of A375 cells. (a) Control A375 cells. (b) Cells were treated with 15 μg/mL concentration of doxorubicin. (c) Cells were treated with 15.62 μg/mL concentration of LDV liposomal formulation of PSI. (d) Comparative bar diagram of apoptotic death after treatment with 15 μg/mL concentration of doxorubicin and LDV liposomal PSI.

FIG. 6: Images of TUNEL assay shows that LDV liposomal formulation of PSI (15.62 μg/mL) increases the amount of fragmented DNA in B16F10 cells after treatment of LDV liposomal formulation of PSI.

FIG. 7: Bar diagram of TUNEL assay shows that LDV liposomal formulation of PSI (15.62 μg/mL) increases the amount of fragmented DNA in B16F10 cells after treatment of LDV liposomal formulation of PSI.

FIG. 8: Body weight of C57BL6J mice was recorded for 90 days after treated with LDV liposomal formulation of PSI.

### DETAILED DESCRIPTION OF THE INVENTION

The present invention relates to a novel LDV liposomal formulation of a natural product being a plant photosystem-I (PSI) and the process of preparation of the said LDV formulation. The novel LDV-peptide based liposomal formulation of PS1 is for specifically targeting α4β1-integrin receptor of melanoma cells (both mouse and human). The preparation of lipopeptide, LDV-long chain and formulation of LDV long chain containing liposome encapsulated PSI is novel. Further, the preparation cost is low and the formulation is highly water soluble.

Since, LDV can specifically bind with α4β1-integrin receptor of melanoma cells, the water soluble LDV-liposomal formulation of PSI can specifically target melanoma cells without affecting normal cells and can solve the issues involved of non-specificity in cancer treatment, which causes whole body toxicity. Therefore, this formulation can be used as an affordable and low cost melanoma therapy.

Isolation of PSI from Spinach:

The photosystem-I (PSI) in the invention is particularly obtained from an easily available plant viz. spinach (Spinacia Oleracea) leaves purchased from Jadavpur market, West Bengal, India, Kolkata-700032. To isolate PS-I from spinach, 500 g of this Indian spinach leaves are homogenized in 400 mM sorbitol and 50 mM tricine-KOH (pH=7.8) in water using liquid nitrogen. The mixture is passed through Mica-cloth. The filtrate is centrifuged at 1000 g for 5 minutes. Pellet is re-suspended in 50 mM sorbitol, 5 mM EDTA-NaOH and 50 mM tricine-KOH (pH=7.8). The mixture is centrifuged at 10000 g for 5 minutes. Pellet is re-suspended in small volume of water. This solution contains total chlorophyll. The absorbance (A) of the chlorophyll solution at 664 nm and 648 nm and concentration of total chlorophyll is calculated using the formula [C_{chlorophyll (a+b)}=(5.24×A₆₆₄+22.24×A₆₄₈) μg/mL]. Final concentration of chlorophyll is made up to 0.8 mg/mL in water. Next, 0.8% (w/v) triton-X-100 is added to the chlorophyll solution and stirred for 30 minutes at room temperature to disperse the membranes of plastid. Plasmid detergent mixture centrifuged at 42000 g for 30 minutes and collects the supernatant. The Supernatant is obtained is loaded on the 25 mL of linear sucrose gradient (0.1M to 2M) and centrifuged at 100000 g at 4° C. for 25 hours. A dark green band present at the interface of 1M and 2M sucrose solution containing the PS-I is collected and stored at −80° C.

### Preparation of LDV Peptide

The trimer LDV peptide [containing amino acids leucine (L), aspartic acid (D) and valine (V) as NH₂-LDV-COOH is synthesized in Microwave Peptide Synthesizer equipped with Liberty 1 system (Discovery, CEM). Wang resin (100-200 mesh) is used as a solid support having substitution level 0.9 mmole/gm. N, N′ dimethyl formamide (DMF) and dichloromethane (DCM) are used as solvent. The amino acids used in the synthesis of LDV are N-terminal fluorenyl methyloxycarbonyl (Fmoc) protected. C₁₆-LDV lipopeptide contains C16 long chain attached on the N-terminal of the tri-peptide with amino acid leucine (L), aspartic acid (D) and valine (V) [i.e. NH₂-LDV-COOH]. This C₁₆-LDV lipopeptide in the liposomal formulation of PS-I aims to target α4β1 integrin. Wang resin is swelled in 1:1 mixture of DMF and DCM solvent for overnight for use. N, N′-di isopropylethyl amine (DIPEA) in DMF having concentration 2M is used as a base and N,N,N′,N′-Tetramethyl-O-(1H-benzotriazol-1-yl)uroniumhexafluorophosphate (HBTU) in DMF having concentration 0.5M was used as an activator. 20% piperidine in DMF was used as de-protection mixture. All the amino acids required for LDV peptide are dissolved in DMF as a concentration of 0.2M separately. After coupling of all the amino acids required for LDV, the N-terminal Fmoc protection was removed by 20% piperidine in DMF. The free —NH2 group of the N-terminal position of the peptide is treated with succinic anhydride and dry triethyl amine (Et₃N) in dry dichloromethane to introduce a carboxyl group at the N-terminal position of the resin. Finally, this terminal —COOH group is coupled with N, N′-di-n-hexadecyl-N-2-aminoethylamine using HOBT and DIC. The resin is dried using DCM, methanol and diethyl ether. Lipopeptide is cleaved from the resin using trifluoro acetic acid (TFA) in DCM. Then, the peptide precipitated out in cold diethyl ether and purified through reverse phase HPLC on a C18 column using MeOH as mobile phase and characterized by Matrix Assisted Laser Desorption Ionization Time of Flight (MALDI-TOF) analysis. The m/z: 936 obtained corresponded to the calculated mass of the LDV lipopeptide (MW=936).

### Preparation of LDV Liposome Containing PSI

The synthesized LDV tripeptide conjugated with C₁₆-long chain prepared as a LDV-liposome, targeted delivery vehicle of PS-I. The formulation contains LDV lipo peptide, DOPC, cholesterol and PSI wherein the ratio of PSI to LDV tri-peptide is in the range of 1:100 to 1:400 is prepared. The thin lipid film prepared by the above mentioned solution mixture is treated with the methanol and chloroform combination with the nitrogen flush method, dried, vacuumed, and finally hydrated with PBS, vortexed, sonicated and stored in freeze at 4° C. References to be made to FIG. 2f regarding the cellular uptake study of the prepared LDV liposome containing PSI in comparison with the prepared controls viz LDV liposome without PSI and normal liposome PSI (without LDV lipo peptide).

### Cell Culture

WI38, B16F10 and A375 cell lines are procured from National Centre for Cell Science (NCCS) Pune, India. Cells are cultured in the lab supplemented with 5% CO₂ humidified atmosphere at 37° C. using dulbecco's modified eagle's medium (DMEM) containing 10% fetal bovine serum, kanamycin sulfate (110 mg/L), penicillin (50 units/mL), streptomycin (50 μg/mL) and trypsin-EDTA (1×) solution for cell splitting.

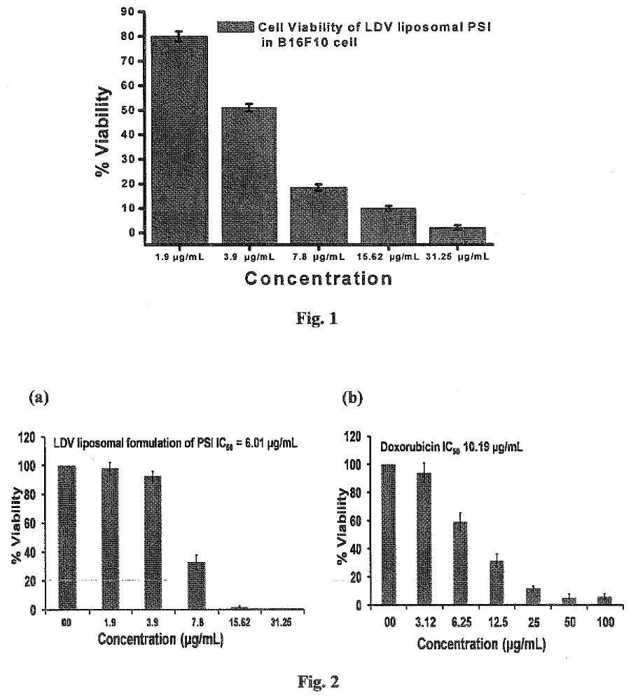

Present invention involves the over-expression of specific integrin receptor proteins on the cell surface of cancer cells (B16F10 and A375) while normal cell (WI38) cannot over-express these proteins. B16F10 melanoma cancer cells over expressed α4β1 integrin receptor on its cell surface and a short tri-peptide Leu-Asp-Val (LDV) can specifically recognize and bind α4β1 integrin receptor. The cell killing efficacy of the formulation was tested using MTT assay in B16F10 and A375 cells. Inventors have observed percentage viability of B16F10 is in the range of 2% to 80% and of A375 melanoma cancer cells is in the range of 0.5% to 94% (FIGS. 1 and 2).

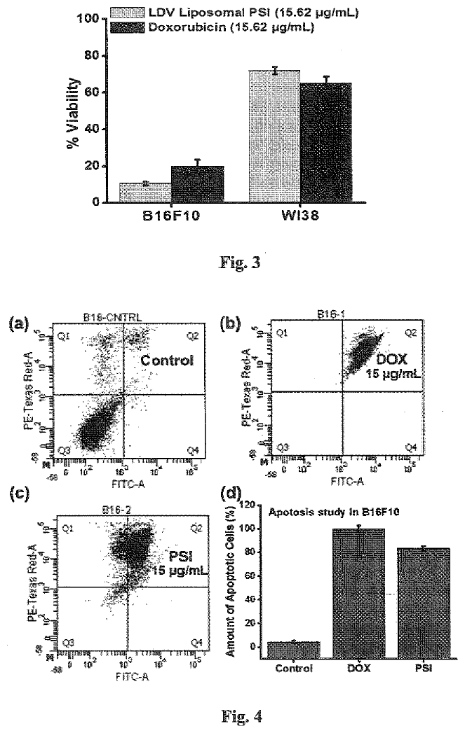

Present investigation shows the cell viability assay of both B16F10 and WI38 cells after treatment with LDV liposome containing PSI and normal liposome containing PSI has been studied and mentioned in Table 5. The study in the Table 5, suggested a significantly high cell death of B16F10 cells, while WI38 cells were unaffected upon treatment with LDV liposome containing PSI whereas both the cell lines were less affected by normal liposome containing PSI. Overall, present investigation indicates that targeted delivery of PSI into the cancer cell keeping normal cell unaffected. Again, the cell viability assay with different concentrations of LDV liposomal formulation of PSI in B16F10 and A375 melanoma cancer cells has been performed and their IC50 values were calculated as described in Table 4. The percent viability of 15.62 μg/mL concentration of LDV liposomal PSI and doxorubicin in the B16F10 and WI38 cell line was compared using MTT assay (FIG. 3). It was found that LDV liposomal PSI has less viability in B16F10 cell but more viability in WI38 cell than doxorubicin. This indicates that LDV liposomal PSI affects cancer cell more and less effective for normal or non-cancerous cell (advantage) whereas doxorubicin is effective for normal or non-cancerous cell too. This is the disadvantage of doxorubicin.

The comparative study of the efficacy of LDV-liposomal formulation of PSI with the marketed drug doxorubicin in human melanoma cancer cell (A375) was performed and the IC50 values were calculated. References may be made to FIG. 2, suggests that the novel LDV liposomal formulation of PSI is highly potent compared to the marketed doxorubicin. As described in the FIG. 2, the LDV-liposomal formulation of PSI has IC50 value (6.01 μg/mL) in A375 cell is significantly less than that of IC50 value (10.19 μg/mL) of doxorubicin. This result indicates that LDV liposomal formulation of PSI is highly potent compared to the doxorubicin. Since significant amount of cell killing was observed by LDV liposomal formulation of PSI, hence the present invention involves understanding the pathway of cell death.

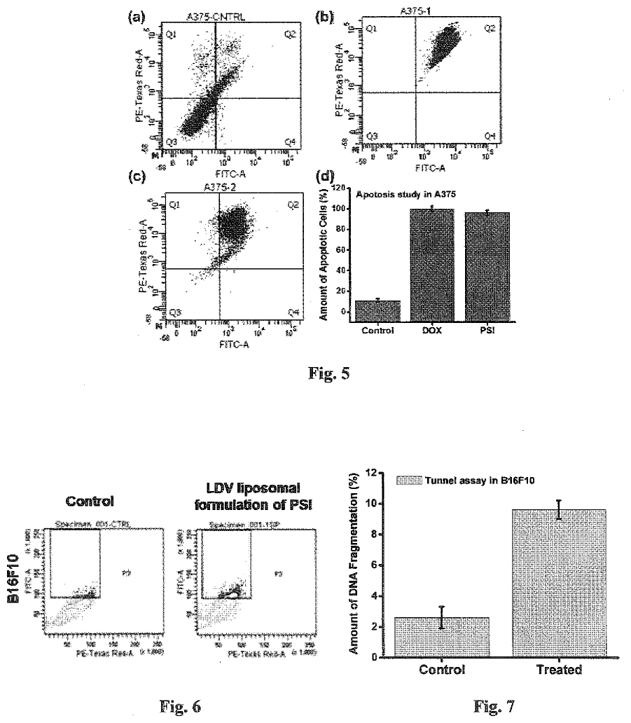

Furthermore FACS analysis using apoptosis detection kit has been performed. FACS analysis, have ascertained that the LDV liposomal formulation of PSI caused apoptotic death of B16F10 and A375 cells (FIGS. 4 and 5). Inventors have performed apoptosis assay using annexin V/PI in B16F10 and A375 cell to compare the amount of apoptotic death of cancer cell due to application of 15.62 μg/mL concentration of LDV liposomal PSI and doxorubicin. Inventors have found large amount of apoptotic death of both the cancer cells (A375 and B16F10) due to LDV liposomal PSI.

It was known that DNA fragmentation was occurred in apoptotic death of cells. Therefore, TUNEL assay was also performed with B16F10 cells after treatment with LDV liposomal formulation of PSI for further confirmation of apoptotic death of cancer cells. Interestingly, it was found that the amount of fragmented or nicked DNA in B16F10 cells was enhanced with respect to the control cells after treatment with PSI (FIGS. 6 and 7). This investigation finally confirmed that LDV liposomal formulation of PSI caused apoptotic death of cancer cells.

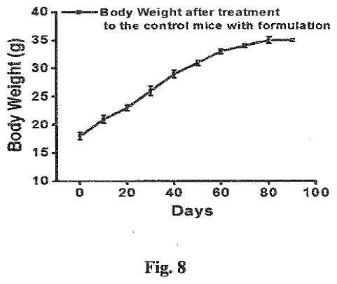

The C57BL6J mice with the LDV liposomal formulation of PSI was treated for 90 days with increasing dose. C57BL6J mice were purchased from National Institute of Nutrition, Hyderabad, India and these mice were housed in the animal house of CSIR-IICB Kolkata. It was found that animals were alive and their body weight also increases. This indicates that the LDV liposomal formulation of PSI is non-toxic to the animals (FIG. 8).

### EXAMPLES

The following examples are given as way of illustration and should not be construed to limit the scope of invention.

### Example 1

### Preparation of LDV Liposome Containing PSI:

Liposome was prepared using DOPC (1 mM), Cholesterol (0.5 mM) and LDV long chain lipo-peptide (1 mM) following hydration method. Solutions of DOPC, cholesterol and LDV long chain lipo-peptide in organic solvent (CHCl₃:MeOH), 9:1 were added in glass vial. A thin film of the lipid mixture was prepared by nitrogen flush. It was dried under vacuum for 6 hours. A 1 mL solution of PSI in phosphate buffer saline (having concentration 62.5 μg/mL) was added into the lipid film for overnight hydration at 4° C. After that it was vortexed for 5 min and then sonicated for 5 minutes at 4° C. Again, it was probe sonicated for 5 times having 30 second pulse on ice bath. Liposome so prepared was stored at 4° C. refrigerator.

### Example 2

### Effect of LDV Liposomal PSI and Normal Liposomal PSI on B16F10 and WI38 Cells:

B16F10 and WI38 cells were seeded in a 96-well plate at density of 10000 cells per well before 24 h of treatment. Cells were treated with LDV liposomal PSI and normal liposomal PSI separately in complete media for 24 h. After 24 h, MTT assay was performed. The greater cell killing by LDV liposomal PSI for the B16F10 cells was observed whereas less cell killing was observed for the WI38 cells in 24 h. In case of normal liposomal PSI, inventors have found similar amount of cell viability for both B16F10 and WI38 cells.

### Example 3

### Cell Viability Assay:

B16F10 and A375 cells were treated with the LDV liposomal formulation of PSI and cellular viability was checked using MTT assay. It is a colorimetric assay where MTT (Methyl thiazolyl diphenyl-tetrazolium bromide), a yellow tetrazole compound, is reduced to purple formazan by cellular reductase enzymes of live cells. Dead cells cannot do this type of reduction and are not able to develop the purple colour. Therefore an idea about the amount of cells lived after the treatment of LDV liposomal formulation of PSI with respect to those cells (control) which are not treated with the peptide. The live cells are called as viable cells and it is denoted as % viability in the Y-axis. On the X-axis four different concentrations of the said formulation was plotted. The absorbance at 550 nm was measured and the % viability was calculated.

Cells were seeded at a density of 10000 cells per well in 96-well plate before 24 hours of treatment. Cells were treated with different concentrations of LDV liposomal formulation of PSI. After that cells were incubated for 48 hours in complete media. MTT solution (5 mg/mL) was prepared in PBS. 50 μL of MTT solution was added into each well and incubated at 37° C. for 4 hours. Purple colored formazan was dissolved in 1:1 (v/v) DMSO/MeOH and absorbances of the wells were measured at 550 nm by micro-plate ELISA reader. The % viability from this absorbance values was calculated. Data depicts the decrease of the % viability with the increase in the concentration of PSI i.e. LDV liposomal formulation of PSI induces the death of cancer cells.

% Viability=[(A550Treated Cells−A550 Backgrounds)/(A550Untreated Cells−A550 Backgrounds)]*100.

LDV liposome without PSI and normal liposome PSI (without LDV lipo peptide) were prepared as controls.

TABLE 1
(Graphical presentation is given in FIG. 1)

| Cell line | Sample | % Viability |
|---|---|---|
| B16F10 | LDV liposomal PSI (31.25 μg/mL) | 2 |
| B16F10 | LDV liposomal PSI (15.62 μg/mL) | 10 |
| B16F10 | LDV liposomal PSI (7.8 μg/mL) | 18.5 |
| B16F10 | LDV liposomal PSI (3.9 μg/mL) | 51 |
| B16F10 | LDV liposomal PSI (1.9 μg/mL) | 80 |

TABLE 2
(Graphical presentation is given in FIG. 2a)

| Cell line | Sample | % Viability |
|---|---|---|
| A375 | LDV liposomal PSI (31.25 μg/mL) | 0.5 |
| A375 | LDV liposomal PSI (15.62 μg/mL) | 1.73 |
| A375 | LDV liposomal PSI (7.8 μg/mL) | 32.7 |
| A375 | LDV liposomal PSI (3.9 μg/mL) | 92.7 |
| A375 | LDV liposomal PSI (1.9 μg/mL) | 94 |

TABLE 3
(Graphical presentation is given in FIG. 2b)

| Cell line | Sample | % Viability |
|---|---|---|
| A375 | Doxorubicin (100 μg/mL) | 6.09 |
| A375 | Doxorubicin (50 μg/mL) | 4.9 |
| A375 | Doxorubicin (25 μg/mL) | 11.7 |
| A375 | Doxorubicin (12.5 μg/mL) | 31.5 |
| A375 | Doxorubicin (6.25 μg/mL) | 59.2 |
| A375 | Doxorubicin (3.12 μg/mL) | 94 |

TABLE 4
Comparison of IC₅₀ values

| | Cell | | |
|---|---|---|---|
| Compound | Line | Type | IC₅₀(μg/mL) |
| LDV liposomal | A375 | Melanoma (human) | 6.01 |
| PSI | | | |
| Doxorubicin | A375 | Melanoma (human) | 10.19 |
| LDV liposomal | B16F10 | Melanoma (Mice) | 3.98 |
| PSI | | | |
| LDV liposomal | WI38 | Lung (human) | Not applicable as cell |
| PSI | | | viability near to 100% |
| Normal liposomal | B16F10 | Melanoma (human) | Not applicable as cell |
| PSI | | | viability near to 100% |
| Normal liposomal | WI38 | Lung (human) | Not applicable as cell |
| PSI | | | viability near to 100% |

TABLE 5
Cell viability study of 15.62 μg/mL concentration of
LDV liposomal PSI and doxorubicin in B16F10 and WI38 cell
lines were performed and graphically presented in FIG. 1.

| Cell Line | Sample | % Viability |
|---|---|---|
| B16F10 | LDV liposomal PSI (15.62 μg/mL) | 10.6 |
| B16F10 | Normal liposomal PSI (15.62 μg/mL) | 91.1 |
| B16F10 | Doxorubicin (15.62 μg/mL) | 19.9 |
| WI38 | LDV liposomal PSI (15.62 μg/mL) | 71.9 |
| WI38 | Normal liposomal PSI (15.62 μg/mL) | 74.3 |
| WI38 | Doxorubicin (15.62 μg/mL) | 65.2 |

### Example 4: Comparison of Cell Viability of LDV Liposomal Formulation of PSI with Doxorubicin

A375 cells were seeded at a density of 10000 cells per well in 96-well plate before 24 hours of treatment. Cells were treated with LDV-liposome-PSI and doxorubicin for 24 hours. MTT assay as described was performed and IC₅₀ value was calculated using CompuSyn software.

TABLE 6
(Graphical presentation is given in FIG. 2a, b)

| Compound | Cell Line | Type | IC₅₀(μg/mL) |
|---|---|---|---|
| LDV liposomal PSI | A375 | Melanoma (human) | 6.01 |
| Doxorubicin | A375 | Melanoma (human) | 10.19 |

### Example 5: FACS Analysis for Apoptosis

Fluorescence activated cell sorting (FACS) experiment was performed for studying the type of cell death. The cell death pathway of B16F10 cells in FACS using apoptosis detection kit was studied. Cells were seeded at a density of ˜5×10⁵ cells per well in a 6-well plate before 24 hours of treatment. The cells were treated with solutions of LDV liposomal formulation of PSI. After that cells were incubated for 48 hours. Cells were trypsinized and washed with PBS by help of centrifugation. Cells were incubated in dark at 37° C. for 30 minutes with a 100 μL solution of assay buffer containing 2.5 μL of Propidium iodide (PI) and 2.5 μL of annexin V. All these reagents were supplied along with the apoptosis detection kit (Santa Cruz Biotechnology). The stock concentration of Propidium iodide and annexin V was 50 μg/mL and 200 μg/mL respectively. After this another 400 μL of assay buffer was added to the cells and these total 500 μL of cell solution was analyzed by FACS. The emission of Annexin V and PI was detected in the FITC and PI channels of BD LSRFORTESA flow cytometer using emission filters at 530 and 610 nm respectively. In the represented data, cells in the Q1, Q2 and Q4 quadrants are regarded as necrotic, late apoptotic and early apoptotic cells respectively. Cells in the Q3 quadrant are regarded as normal cells or healthy cells. Data was analyzed using FACS DIVA software. The control cells (cells were not treated with PSI) were mostly populated in Q3 quadrant. Population of cells in the Q4 and Q2 quadrant was increased when cells were treated with LDV liposomal formulation of PSI. The comparative study of the percentage of healthy and apoptotic cells after treatment with different concentrations of PSI with the control (untreated cell) was done using a bar diagram.

TABLE 7
(Graphical presentation is shown in FIG. 4 d)

| | | Amount of Apoptotic |
|---|---|---|
| Cell Line | Sample | death of cancer cell (%) |
| B16F10 | Control | 4.3 |
| B16F10 | Doxorubicin (15.62 μg/mL) | 99.9 |
| B16F10 | LDV liposomal PSI (15.62 μg/mL) | 83.3 |

TABLE 8
(Graphical presentation is shown in FIG. 5 d)

| | | Amount of Apoptotic |
|---|---|---|
| Cell Line | Sample | death of cancer cell (%) |
| A375 | Control | 11.1 |
| A375 | Doxorubicin (15.62 μg/mL) | 100 |
| A375 | LDV liposomal PSI (15.62 μg/mL) | 96.3 |

### Example 6: TUNEL Assay for Specific Detection of Apoptosis

B16F10 cells were seeded in a 6-well plate at density of ˜5×10⁵ cells per well before 24 h of treatment. Cells were treated with LDV liposomal formulation of PSI. After that cells were incubated for 24 h in complete media. Cells were washed with phosphate buffer and trypsinized. Cells were centrifuged with PBS at 3000 rpm for 3 minutes. Cell pellet was treated with ice cold 1% para formaldehyde solution in PBS and incubated for 20 minutes at 4° C. Cells were centrifuged at 3500 rpm for 4 minutes. Cell pallet was dissolved in 200 μL of PBS and then cells were fixed with 1000 μL of ice cold EtOH (100%). Cells were stored at −20° C. before experiment. Fixed cells were centrifuged at 3000 rpm for 5 min. Cell pallet was incubated with 100 μL of equilibration buffer (supplied along with the kit) for 8 minutes. Inventors have prepared nucleotide mixture on ice tub using 45 μL of equilibration buffer, 5 μL of nucleotide mix and 1 μL of rTdT enzyme for each cell pallet. In case of unstained sample inventors have not used the rTdT enzyme during preparation of nucleotide mixture. Cell pallet was treated with 50 μL of the nucleotide mixture and incubated at 37° C. water bath for 90 minutes in dark. Cells were treated with 1000 μL of 20 mM EDTA solution (prepared in water) and vortexed. Cells were centrifuged at 4000 rpm for 5 minutes. Cell pallet was treated with 400 μL solution of working PI solution containing PI (100 μg/mL) and RNaseA (10 μg/mL) and incubated for 30 minutes at room temperature. Cells were analyzed by flow cytometer.

TABLE 9
(Graphical presentation is given in FIG. 7)

| | | Amount of DNA |
|---|---|---|
| Cell line | Sample | fragmentation (%) |
| B16F10 | Control | 2.6 |
| (mouse melanoma) | | |
| B16F10 | LDV liposomal PSI | 9.6 |
| (mouse melanoma) | (15.62 μg/mL) | |

### Example 7: Toxicity Study of LDV Liposomal Formulation of PSI

The toxicity study of LDV liposomal formulation of PSI on the C57BL6J mice model was performed. C57BL6J mice were treated with the LDV liposomal formulation of PSI for 90 days with increasing dose (up to 100 μg/mL). It was found that animals were alive and their body weight also increased.

TABLE 10
(Graphical presentation is given in FIG. 8)

| Days | Body Weight (gm) of mice | Error (gm) |
|---|---|---|
| 0 day | 18 | 0.6 |
| 10 days | 21 | 0.7 |
| 20 days | 23 | 0.5 |
| 30 days | 26 | 0.8 |
| 40 days | 29 | 0.7 |
| 50 days | 31 | 0.5 |
| 60 days | 33 | 0.4 |
| 70 days | 34 | 0.3 |
| 80 days | 35 | 0.6 |
| 90 days | 35 | 0.2 |

### Advantages:

The main advantages are:
1. Present invention provides a novel formulation of natural product, LDV liposomal formulation of PSI, as an anti-cancer agent.
2. LDV liposomal formulation of PSI can be prepared easily and is easily available compared to the other marketed anti-cancer drugs.
3. It costs very little to isolate PSI from green leaves and isolation process is very easy.
4. The marketed cost of PSI will be very cheap compared to the known marketed anti-cancer drugs.
5. LDV liposomal formulation of PSI should have fewer side effects.
6. LDV liposomal formulation of PSI specifically kills the cancer cells.
7. The main problem with the known anticancer drug is bioavailability. LDV liposomal formulation of PSI being water soluble reduces such problem.

### REFERENCES

1. Newman, D. J., and Cragg, G. M. (2012) Natural products as sources of new drugs over the 30 years from 1981 to 2010. Journal of Natural Products 75, 311-335. (Ref: 7)
2. Cragg, G. M., and Newman, D. J. (2005) Plants as a source of anti-cancer agents. Journal of Ethnopharmacology 100, 72-79. (Ref: 8)
3. Jordan, M. A., Thrower, D., and Wilson, L. (1991) Mechanism of Inhibition of Cell Proliferation by Vinca Alkaloids. Cancer Research 51, 2212-2222. (Ref: 9)
4. Nobler, L. (1990) The discovery of the vinca alkaloids-chemotherapeutic agents against cancer. Biochem. Cell Biol. 68, 1344-1351. (Ref: 10)
5. Amunts, A., Drory, O., and Nelson, N. (2007) The structure of a plant photosystem I supercomplex at 3.4 A resolution. Nature. 447, 58-63.
6. Tjus, S. E., Scheller, H. V., Andersson, B., and Møller, B. L. (2001) Active oxygen produced during selective excitation of photosystem I is damaging not only to photosystem I, but also to photosystem II. Plant Physiol. 125, 2007-15.
7. Mao, X., Yu, C. R., Li, W. H., and Li, W. X. (2008) Induction of apoptosis by shikonin through a ROS/JNK-mediated process in Bcr/Abl-positive chronic myelogenousleukemia (CML) cells. Cell Res. 18, 879-88.
8. Bellemare, F., Boucher, N., and Lorrain, L. (2010) Thylakoids and their functional derivatives, and their uses. Patent number WO2010085876 A1, PCT/CA2010/000094.
9. Pennisi, P. C., Jensen, E. P., Zachar, V., Greenbaum, E. and Yoshida, Ken, (2009) Incorporation of Photosynthetic reaction centers in the Membrane of Human Cells: Towards a New Tool for optical Control of Cell Activity. Cellular and Mol. Bioengg., 2, 156-165.
10. Kiley, P., Zhao, X., Vaughn, M., Baldo, A. M., Bruce, D. B., and Zhang, S. (2005) Self-Assembling Peptide Detergents Stabilize Isolated Photosystem I on a dry Surface for an Extendent Time. PLoS Biology, 3(7), e230.
11. Ledezma, E., Castro, R. A., and Cardier, J. (2004) Apoptotic and anti-adhesion effect of ajoene, a garlic derived compound, on the murine melanoma B16F10 cells: possible role of caspase-3 and the alpha(4)beta(1) integrin. Cancer Lett. 206, 35-41. (Ref: 33)
12. Zhong, S., Bhattacharya, S., Chan, W., Jasti, B., and Li, X. (2009) Leucine-Aspartic Acid-Valine Sequence as Targeting Ligand and Drug Carrier for Doxorubicin Delivery to Melanoma Cells: In Vitro Cellular Uptake and Cytotoxicity Studies. Pharmaceutical Research 26, 2578-2587. (Ref: 34)
13. Hua, Z. J., and Xu, M. (2000) DNA fragmentation in apoptosis. Cell Research 10, 205-211. (Ref: 32)

1. A novel LDV liposomal formulation of photosystem-1 for anticancer therapy, wherein the ratio of photosystem-I to LDV tri-peptide conjugated with C₁₆-long chain is in the range of 1:100 to 1:400.

2. The LDV liposomal formulation of photosystem-1 as claimed in claim 1, wherein the liposome comprises of LDV tri-peptide conjugated with C₁₆-long chain, 1,2-Dioleoyl-sn-glycero-3-phosphocholine and cholesterol in a ratio of 1:1:0.5 respectively.

3. The LDV liposomal formulation of PS1 as claimed in claim 1, wherein the percentage viability of B16F10 is in the range of 2 to 80%.

4. The LDV liposomal formulation of PS1 as claimed in claim 1, wherein the percentage viability of A375 melanoma cancer cells is in the range of 0.5 to 94%.

5. The LDV liposomal formulation of PS1 as claimed in claim 1, wherein IC₅₀ value of the said formulation is found to be in range of 3.98 to 6.01 μg/mL for B16F10 and A375 melanoma cancer cells.

6. The LDV liposomal formulation of photosystem-I as claimed in claim 1, useful for causing 83.3% and 96.3% apoptotic death of B16F10 and A375 cancer cells respectively at a concentration of 15 μg/mL.

7. A method of preparation of the LDV liposomal formulation of photosystem-I as claimed in claim 1, wherein the method comprises of the following steps of:
a) mixing chloroform in a glass vial, with LDV-C16 long chain (1 mM), DOPC lipid (1 mM) and cholesterol (0.5 mM) to obtain a solution,
b) removing chloroform from the mixture obtained in step (a) followed by drying the vial by evaporation using nitrogen flash,
c) drying the vial under vacuum for 2 hours to obtain dried mixture,
d) adding PSI to the dried mixture obtained in step (b) followed by incubation for a period in the range of 6 to 8 hrs at 4° C. vial and incubating it overnight, at 4° C.,
e) vortexing the solution in the vial by bath sonicating and probe sonicating.

8. The LDV liposomal formulation of photosystem-I as claimed in claim 1, wherein the prepared formulation obtained from an isolate of an edible plant is less toxic compared to the anti-cancer drugs selected from a group of doxorubicin, dasatinib.
